SEQUENCE LISTING (3 sequences)

SEQ ID NO: 1 - protein, 89 aa, Homo sapiens
MFLVHSSNNFGAILSSTNVEAEEDGDLQCLCVKTTSQVRPRHITSLEVIKAGPHCPTAQLIATLKNGRKICLDLQAPLYKKIIKKLLES

SEQ ID NO: 2 - protein, 108 aa, Homo sapiens
MFLVHSSNNFGAILSSTNVEAEEDGDLQCLCVKTTSQVRPRHITSLEVIKAGPHCPTAQLIATLKNGRKICLDLQAPLYKKIIKKLLESFLVHSSNNFGAILSSTNVE

SEQ ID NO: 3 - protein, 872 aa, Homo sapiens
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVNVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2012/0040373 A1
(43) Pub. Date: Feb. 16, 2012

(54) Integrated Diagnosis of Heparin-Induced Thrombocytopenia

(76) Inventor: Mariano Ubeda, Natick, MA (US)

(21) Appl. No.: 13/072,214

(22) Filed: Mar. 25, 2011

Related U.S. Application Data
(60) Provisional application No. 61/373,336, filed on Aug. 13, 2010.
(60) Provisional application No. 61/412,636, filed on Nov. 11, 2010.
(60) Provisional application No. 61/423,344, filed on Dec. 15, 2010.

(51) Int. Cl. G01N 33/566; G01N 33/543; G01N 33/53

(52) U.S. Cl. 435/7.21; 436/501; 436/518

(57) ABSTRACT

Disclosed are methods for the early detection of heparin-induced thrombocytopenia comprising the quantitative detection of chemokine platelet factor-heparin complexes, autoantibodies to such complexes, and platelet activation. Also disclosed are assays to detect the propensity of a subject to develop heparin-induced thrombocytopenia.

### CROSS-REFERENCE TO RELATED APPLICATIONS

This application claims the benefit of U.S. Provisional Patent application Ser. No. 61/373,336, entitled “Detection and Monitoring of Inflammatory and Autoimmune Chronic Diseases,” which was filed Aug. 13, 2010; U.S. Provisional Patent application Ser. No. 61/412,636, entitled “Integrated Approach for Laboratory Diagnosis of Heparin-Induced Thrombocytopenia (ID-HIT),” which was filed on Nov. 11, 2010; and U.S. Provisional Patent Application Ser. No. 61/423,344, entitled “Integrated Approach for Laboratory Diagnosis of Heparin-Induced Thrombocytopenia (ID-HIT),” which was filed on Dec. 15, 2010. The entirety of the aforementioned applications is herein incorporated by reference.

### FIELD OF INVENTION

This invention is in the field of medical diagnostics, and in particular, relates to the early diagnosis and detection of heparin induced thrombocytopenia.

### BACKGROUND

Heparin-induced thrombocytopenia (HIT) is a heparin-induced prothrombotic process that can result in formation of intravascular clots (thrombosis) and secondary ischemic consequences. Ischemia then produces organ damage such as tissue necrosis, stroke, heart attack, and amputations with potential fatal consequences. The diagnosis of HIT is very difficult and complicated by the fact that it mainly occurs in hospitalized patients undergoing complex medical and surgical treatments and therefore already afflicted by other serious medical conditions. The pathogenic mechanism responsible for HIT involves the formation of antibodies that recognize a complex between heparin and the chemokine platelet factor 4 (PF4, also known as CXCL4). Typically, HIT patients have been treated with heparin for a period of 5 to 20 days before they develop thrombocytopenia and thrombotic complications with no other apparent reason. Patients that have previously being exposed to heparin may develop HIT as little as five days after initiation of heparin administration. There is also a form of HIT that develops in patients that have already stopped heparin therapy. This delayed-onset form of HIT is accompanied by potentially fatal thrombotic complications and occurs on average 5-19 days after heparin discontinuation.

HIT is due to the production of autoantibodies that recognize PF4 and heparin complexes (PF4-H). The resulting immunocomplexes bind the plasma membrane of platelets by interactions with FcγIIa receptors. This, results in receptor aggregation within specific regions of the plasma membrane denominated lipid rafts that contribute to its activation. Activation of FcγIIa receptors within the lipid rafts serves to recruit other downstream signaling proteins. One of these proteins is the protein kinase SyK. Binding and activation of SyK is responsible for translating the information downstream and eventually induce the activation of platelets, their aggregation, and the formation of clots within the vascular system. As a result, intravascular thrombi are produced due to ischemia and necrosis of different organs. The consequences may range from life-threatening conditions such as myocardial infarction, pulmonary embolism (PI), and stroke, to milder forms of thrombosis such as deep vein thrombosis (DVT) and skin necrosis. Thrombocytopenia seems to be only a secondary consequence of platelet consumption and rarely is severe enough as to produce clinical consequences by itself.

Since this is an immunological reaction, even exposure to low doses of heparin, such as the amount contained on medical devices (e.g., endovascular catheters), may be sufficient to trigger HIT. Although heparin-PF4 (H-PF4) autoantibodies are usually present in patients with HIT, the autoantibodies can be present in a large proportion of patients treated with heparin that never develop HIT. Individuals susceptible to develop HIT include patients exposed to unfractionated heparin (UFH), low molecular weight heparin (LMWH), and a synthetic pentasaccharide heparin analog (fondaparinux) and other heparin-like molecules.

In HIT, circulating autoantibodies react with heparin-PF4 complexes that are recognized by Fc receptors (FcγRIIa) located in the plasma membrane of the platelets. This induces platelet activation, the release of serotonin and other granule components, the formation and release of microparticles that assist in the activation of the coagulation cascade, and morphological changes in the platelets that support aggregation and clot formation. Many patients develop moderate thrombocytopenia without the accompanying thrombosis probably due to destruction of platelets in the spleen without significant aggregation and thrombosis. On the other hand, some patients may develop thrombosis without the accompanying thrombocytopenia. Therefore, platelet count, alone, is not a good marker for HIT as it does not reflect platelet activation and the formation of thrombi.

Thus, what is needed is the early prediction and detection of those patients that will develop thrombotic complications and the ability to distinguish them from those afflicted by moderate thrombocytopenia that do not develop harmful thrombosis.

Current diagnosis of HIT is mainly based on clinical findings. There are several laboratory diagnostic assays available. However, their role is only to corroborate and confirm the clinical observations. These assays do not provide information in time for it to be considered in the decision-making process, and in others the information is not specific enough to be considered relevant for the decision. For these reasons, the diagnosis of HIT is mainly dependent on clinical findings. A systematic approach to score these findings was developed and denominated 4T's score (Thrombocytopenia, Timing, Thrombosis, and absence of other causes of Thrombocytopenia) is widely used to distinguish HIT from other causes of thrombocytopenia (Lo et al. (2006) J. Thromb. Haemost. 4(4):759-765). Based in this score system the probability of suffering from HIT is classified as high (6-8 points), intermediate (4-5 points), or low (<3 points).

There are two major types of laboratory tests available for HIT: a classical bioassay also known as functional platelet assays; and immunoassays detecting the presence of autoantibodies recognizing heparin-PF4 in circulation. The serotonin release assay is presently considered the gold standard for HIT diagnosis. This assay directly tests the effect of the patient serum on isolated platelets. However, this bioassay is not specific for HIT, because any circulating immunocomplex is capable of inducing a positive response. Furthermore, the serotonin release assay (SRA) is not commercially available, requires highly trained personnel and can only be performed in very specialized reference laboratories. On average it takes between 4-6 days for the clinician to get the results from this test, a period that is too long for patient susceptible to suffer life-threatening consequences to wait until a therapeutic decision can be made.

Other, more sensitive methods for the detection of the HIT autoantibodies also exist. However, the fact that these assays are sensitive creates an additional problem in the diagnosis of HIT: the majority of patients that develop autoantibodies never end up developing HIT. The rate of false positives for these ELISA assays depend on the conditions of each particular group of patients, but it can reach up to 60% in patients undergoing cardiopulmonary bypass surgery. Improvement on their specificity has been made by focusing specifically on anti-PF4-H IgG antibodies due to the fact that recognition and binding of the immunocomplexes to platelets involves the IgG-specific FcγR-IIa receptor

In spite of these improvements, the diagnosis of HIT and therefore the decision of therapeutic intervention (e.g., substituting heparin for a direct thrombin inhibitor such as argatroban, lipidurin or danaparoid) are mostly based on the proper interpretation of clinical findings which rely on the degree of experience of individual physicians. Even the combination of data from ELISA and serotonin release assays only serves to confirm the diagnosis, and to assure that the right approach was taken.

Thus, what is needed is a new approach to improve the diagnosis of HIT and to provide the clinicians with a reliable diagnostic tool to help in the decision process and allow earlier interventions to prevent the development of thrombotic complications.

### SUMMARY

Disclosed is a new method for the early detection of HIT. This assay method (ID-HIT) integrates information derived from the three major components of the pathophysiology of HIT. Information derived from these three different processes is quantified and integrated, for example, using appropriate software in order to provide clinicians with a unique piece of information which allows them to determine whether the patient has, does not have, or has a significant risk to develop HIT and thus if heparin therapy should be initiated. The assay is designed to be used in multiple platforms.

The first component or “Channel 1” provides quantitative information about the formation of heparin-platelet factor 4 complexes or aggregates (H-PF4) free in circulation and attached to the surface of platelets and circulating microparticles. The second component or “Channel 2” provides quantitative information about the formation of autoantibodies against PF4-H complexes and their avidity to bind platelets and microparticles. The third component or “Channel 3” provides information about platelet activation and clot formation linked to the HIT immunocomplexes. The quantitative information provided by each channel can be used together or separately for the evaluation of other aspects of HIT diagnosis, such as the identification of the best available therapeutic agents, the determination of the most appropriate time to initiate the therapeutic intervention, as well as the follow-up and monitoring of the progression of the underlying pathological process.

In one aspect, the disclosure provides a method for the detection of HIT in a mammalian patient, comprising: (a) quantitatively measuring the presence of PF4-H complexes in a sample from a patient or the ability to form these complexes in vitro; (b) detecting the presence of pathogenic autoantibodies specific for PF4-H complex in the patient sample: and (c) detecting the presence of platelet activation in the patient. The presence of PF4-H complexes, PF4-H autoantibodies, and platelet activation are indicative of the presence of HIT. The patient may be a mammalian patient, such as a human.

In one embodiment, step (a) is performed prior to treatment of the patient with heparin, during heparin treatment, and post-heparin treatment. In another embodiment, step (a) is performed prior to the patient being treated with heparin, heparin being added to the patient sample before quantitatively measuring the presence of PF4-H complexes.

In another embodiment, the presence of PF4-H complexes is measured in a fluid sample from the patient, such as from blood, serum, plasma, saliva, tears, seminal fluid, sweat, or processed tissue samples such as tissue extracts. In one embodiment, the patient sample is pre-treated with a low stringency detergent to release lipid rafts from the platelets and microparticles, solubilizing membrane bound PF4-H complexes, and release membrane trapped HIT antibodies

In some embodiments, the steps of measuring the presence of PF4-H complexes, of PF4-H autoantibodies, and of platelet aggregation occurs in one reaction solution, or in at least one reaction solution, in three reaction solutions. These three steps may occur. The method may alternatively include steps (a), (b), and (c) occurring simultaneously or sequentially.

In certain embodiments, the presence of PF4-H complexes is measured in-vitro by immonodetection, affinity binding, or fluorescence resonance transfer (FRET). In some embodiments, the immunodetection method is ELISA, lateral flow, or multiplex technology. In particular embodiments, the presence of PF4-H complexes is detected using one or more than one antibody specific for the PF4-H complex, the antibody not recognizing heparin or PF4. In one embodiment, a first antibody recognizes an epitope of PF4 not neutralized by the presence of heparin, and a second antibody recognizes an epitope of heparin not neutralized by the presence of PF4.

In other embodiments, the presence of heparin in the PF4-H complex is recognized by a heparin-binding compound which is not an antibody, including heparin-binding peptides, heparin binding domains, and receptors for heparin. In other embodiments solid support surfaces are conjugated with a heparin-binding molecules (e.g., EpranEx™-BMG Labtech-, and HB-EGF) to capture the PF4-H complexes, and a labeled antibody recognizing PF4 or recognizing PF4-H is used for detection.

In another embodiment, the pathogenic autoantibodies are detected by: (a) creating a genetically engineered PF4 fusion molecule comprising a protein aggregation domain (PAD); (b) adhering the fusion molecule to a solid support; (c) contacting the adhered fusion molecule with the patient sample; and (d) detecting and quantifying antibodies that bind to the adhered fusion protein. In some embodiments, the PF4 fusion protein is prepared using a recombinant PAD-PH4 expression vector. In certain embodiments, the bound autoantibodies are detected with an anti-human immunoglobulin. In some embodiments, the bound autoantibodies are detected with anti-IgG, while in other embodiments the bound antibodies are detected by an affinity-binding compound such as Protein A and Protein G. In other embodiments an affinity binding compound, such as protein A and protein G is use to capture the antibodies in solution, and labeled antigenic PF4-H complex, labeled antigenic aggregated PAD-PF4, or labeled aggregated PAD-PF4-H complexes are used for detection. In other embodiments, solid supports, such as beads, are conjugated with Fc receptors that recognize the Fc domain of the HIT antibodies, and labeled antigenic PF4-H complex, labeled antigenic aggregated PAD-PF4, or labeled aggregated PAD-PF4-H complexes are used for detection.

In some embodiments, the presence of platelet activation in the patient is determined by: (a) immobilizing an antibody specific for PF4-H to a solid support; (b) contacting the immobilized antibody with a sample from the patient, the platelets and/or microparticles in the sample binding to the antibody, thereby clustering activated Fc receptors on the bound platelet and/or microparticle membrane domains; (c) treating the bound membrane with detergent to form a microparticle patch or a platelet patch; and (d) detecting the presence of activated Fc receptor in the bound microparticle patch or in the bound platelet patch, the activated Fc receptor being indicative of the presence of platelet activation in the patient.

In particular embodiments, the activated Fc receptor is measured by detecting SyK protein kinase activity, SyK protein kinase being activated when the Fc receptor is activated. In other embodiments, the SyK protein kinase activity is measured by detecting enzymatic activity, fluorescence, or radioactive labels conjugated with SyK kinase or to its amino-terminal region containing the two SH2 binding domains. In certain embodiments, a detectable label is attached to the SH2 domain of SyK.

In other embodiments, the activated Fc receptor is detected by measuring its binding to a substrate other than SyK kinase such as a specific antibody recognizing the activated intracellular domain of the Fc-receptor or other Fc-receptor associated adaptor proteins such as SLP-65 and SLP-76.

Platelet activation is alternatively measured by: (a) immobilizing CD41 antibodies specific for platelet CD41 to a solid support; (b) contacting the immobilized CD41 antibodies with the patient sample; (c) detecting the presence of microparticles from the patient sample adhered to the immobilized CD41 antibodies with: (i) an anti-PF4-H antibody; (ii) a phosphatidylserine (PS) binding molecule; and/or (iii) an anti-tissue factor antibody, the bound microparticles being indicative of platelet activation. In certain embodiments, the PS binding molecule comprises an anti-PS antibody or annexin V. In particular embodiments, the platelets and microparticles are separated by a method not involving CD41. In some embodiments, other platelet-specific membrane markers, such as CD42b or CD31 are used in the separation process, may also include physical separation and isolation by size-specific filtration of platelets and microparticles.

In an alternative embodiment, platelet activation is measured by: (a) immobilizing a peptide containing the SH2 domain sequence of SyK to a solid support; (b) contacting the immobilized SH2 domain of SyK with the patient sample; (c) detecting bound platelet patches and bound microparticle patches by contacting the platelet and microparticles patches adhered to the SH2 domain of SyK with: (i) an antibody recognizing PF4-H complexes; (ii) an antibody recognizing PF4; (iii) an antibody recognizing heparin; or (iv) a non-antibody binding compound which recognizes heparin and/or PF4.

### DESCRIPTION OF DRAWINGS

The foregoing and other objects of the present invention, the various features thereof, as well as the invention itself may be more fully understood from the following description, when read together with the accompanying drawings in which:

FIG. 1 is a diagrammatic representation of the pathophysiology of heparin induced thrombocytopenia;

FIG. 2 is a diagrammatic representation of the Iceberg Model of heparin induced thrombocytopenia adapted from Warkentin et al. (Ann. Throac. Surg. (2007) 83: 21-23);

FIG. 3 is a diagrammatic representation of an integrated diagnostic method of the present disclosure. This method evaluates the activity of the different pathophysiological elements of the pathophysiology of HIT, which represent sequential steps of the HIT pathophysiologic process.

FIG. 4A is a diagrammatic representation of an embodiment of Channel 1 of the present assay which uses PH4-H monoclonal antibody on a solid support to capture the complexes present in the patient sample, and then uses a second labeled antibody against a different epitope of the complex for detection and quantification of the complexes concentration;

FIG. 4B is a diagrammatic representation of another embodiment of Channel 1 of the present assay in which PF4-H complexes are detected after incubation of the patient sample with heparin to evaluate patient predisposition to form heparin-PF4 complexes and serves as an initial indication of individual patient risk to suffer HIT prior to heparin exposure;

FIG. 5A is a diagrammatic representation of an embodiment of the method utilized for detection and quantification of pathogenic HIT auto-antibodies (Channel 2), where the antigenic component is made of aggregated PF4 that has been modified through the addition of a peptide domain responsible for protein association, the new human hPAD-PF4 fusion protein being conjugated to a solid surface and acts as a surrogate epitope for HIT antibodies for use in Channel 2;

FIG. 5B is a diagrammatic representation of another embodiment of the method of invention utilized to generate an antigenic target for HIT autoantibodies quantification where heparin is used during the process of aggregation in order to accelerate the process of hPAD-PF4 folding and to increase the specificity of pathogenic HIT antibodies detection;

FIG. 5C is a representation of the process of forming the surrogate epitope from the aggregation of the fusion protein containing one protein association domain at the amino-terminal domain of PF4;

FIG. 5D is a schematic representation of the amino acid sequence of a representative fusion protein used to make the monomer of a representative surrogate epitope having one PAD, which is made up of a 20 amino acid sequence from the human Islet Amyloid Polypeptide (hIAPP) (Human IAPP SEQ ID: P10997, aa 49-67 (in yellow) which is the minimal domain sequence required for self-aggregation and the sequence of the human PF4 (hIAPP^{PAD}-PF4 fusion protein)), and the whole sequence (aa 32-101) for matured PF4 (Human PF4 (also CXCL4) SEQ ID: P02776 (in red);

FIG. 5E is a diagrammatic representation of the preparation of an engineered PF4 aggregate that serves as surrogate target epitope for HIT antibodies for Channel 2, where the monomer making up the epitope aggregate has two PADs, the presence of the two PADs accelerating the process of PF4 aggregation and proper folding of epitopes in the aggregate;

FIG. 5F is a schematic representation of the amino acid sequence of a representative double fusion construct (hIAPP^{PAD}-PF4) (sequence as in FIG. 5D but with two repetitive P10997 self-aggregation sequences (SEQ ID: NO. P10997 (aa 49-67 in yellow) once placed at the amino-terminal end and one placed at the carboxy-terminal end of the PF4 sequence, and PF4 (also CXCL4) having SEQ ID: P02776 (aa 22-101 in red), used to increase the aggregation avidity of the monomer of a representative surrogate target epitope (shown in FIG. 5E);

FIG. 5G is a diagram showing three representative currently used methods to create antigenic targets for the detection of HIT antibodies in patients suspected to suffer from HIT, where (A) has been developed and/or commercialized by Stago (Asnières sur Seine, France), (B) GTI (Waukesha, Wis., USA), and (C) Hyphen (Neuville-sur-Oise, France);

FIG. 6A is a diagrammatic representation of an embodiment of Channel 3 of the present assay utilizing lipid rafts of platelet and/or platelet-derived microparticles to evaluate the level of platelet activation associated with HIT, wherein both antibody recognition of the H-PF4 complex on the outer surface and active Fc receptor domains on the inner surface are used in a sandwich-like assay, and where one method for recognition of active phosphorylated FcR gamma (ITAM domain) involves the specific binding of SyK kinase with the active FcR through specific SH2 domains;

FIG. 6B is a schematic representation of the amino acid sequence of a fusion protein containing SyK (Human SyK SEQ ID: P43405, aa 1-635 in black) in which the SH1 and SH2 domains required for Fc interaction are underlined (aa 1-259) and a fluorescent label (e.g., green-fluorescent protein GFP (SEQ ID: P42212 in green) which is a representative method used for detection and quantification of activated FcR associated with lipid rafts previously immobilized though antibodies recognizing H-PF4 complexes;

FIG. 6C is a diagrammatic representation of several useful targets of the functional protein domains of the SH2-SyK construct (FIG. 6B) where (1) is the long construct containing ATP, kinase, and GFP domains; (2) is the short construct containing a GFP domain; and (3) is the binding-only sequence;

FIG. 6D is a diagrammatic representation of the procedure for detection of platelet activation associated with HIT (channel 3), where a PF4-H monoclonal antibody recognizes H-PF4 complexes associated with platelets and microparticles, and a labeled recombinant protein containing the SH2 domain of SyK serves for detection and quantification by binding phosphorylated FcR gamma (ITAM domains) previously activated by the immunocomplexes and where a low stringency detergent is used to disrupt the membrane of platelets and microparticles in order to gain access to the inner membrane surface;

FIG. 6E is a diagrammatic representation of another exemplary method utilized for detection of platelet activation associate with HIT, where membrane disruption is performed early in the process before the initial binding step, and where binding to the solid surface occurs through recognition of activated Fc receptors in lipid rafts by a peptide (short construct in FIG. 6C) containing the SH2 domains of SyK previously conjugated to the microspheres or other solid surface;

FIG. 6F is a representation of another embodiment of a method to detect and quantify platelet activation associated with HIT (Channel 3), where platelet-derived microparticles are recognized by microspheres (or another solid support) previously conjugated with an antibody recognizing the platelet specific CD41 molecule, and detection is then performed using labeled monoclonals antibodies recognizing the H-PF4 complex, annexin V to recognize the phosphatydylserine component in microparticles and an antibody recognizing tissue factor (TF);

FIG. 7A is a graphic representation of a sample assay data from each of the three pathophysiological elements of HIT (Channels 1, 2, and 3) from pre-heparin exposure to post-heparin exposure for a typical HIT case (A) and for a negative control patient (B);

FIG. 7B is a graphic representation of sample assay readings from heparin expose for a high risk patient with prior exposure (1) and for a high risk patient with inflammation and a history of extensive surgery (2);

FIG. 8 is a graphic representation of the projective information on the evolution of the pathogenic process in a particular subject derived from at least two ID-HIT assays; and

FIG. 9 is a schematic representation of a simplified representative procedure for performing the ID-HIT assay using a multiplex reading system such as the Luminex xMAP, where after the pre-treatment step, the sample is split in two aliquots and processed separately in order to avoid beads/beads interference.

### DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

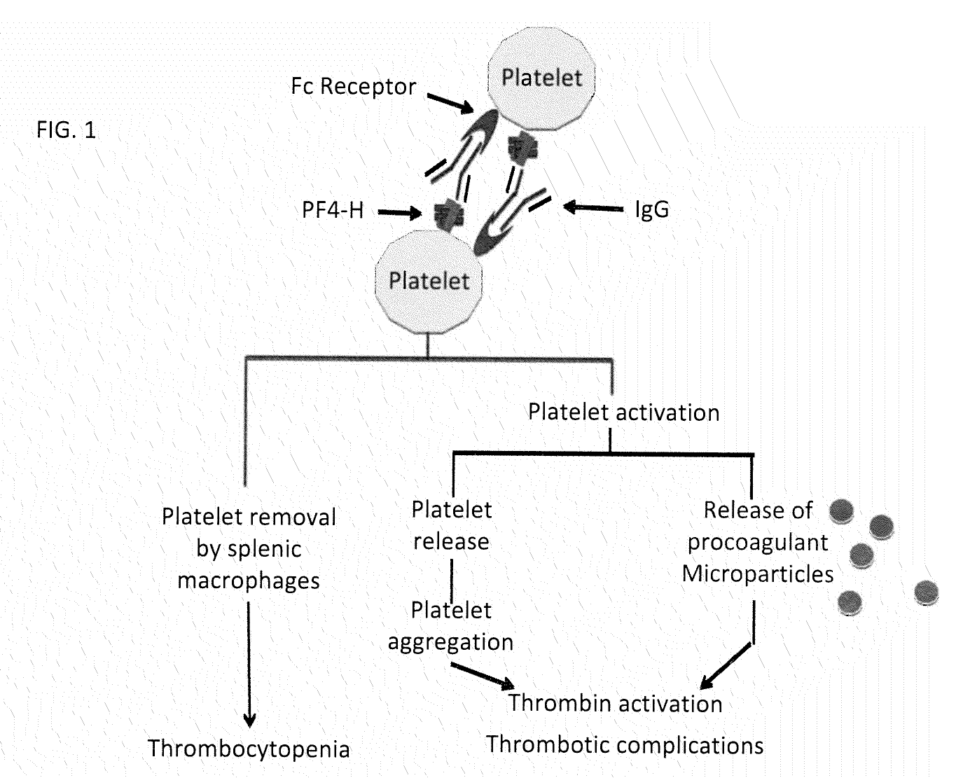

In HIT, activation of platelets and aggregation is a consequence of the presence of autoantibodies that recognize epitopes on the endogenous platelet factor 4 (PF4) created through its interaction with exogenous administered heparin (H). The resulting PF4-H complexes can be found free in circulation or localized to the plasma membrane of platelets, microparticles, and endothelial cells. Recognition of the PF4-H complex by platelet Fc receptors induces platelet activation and the further release of PF4 into the circulation. Platelet activation results in platelet aggregation and the formation of thrombi, which depletes the concentration of circulating platelets (thrombocytopenia). This pathology is shown in FIG. 1.

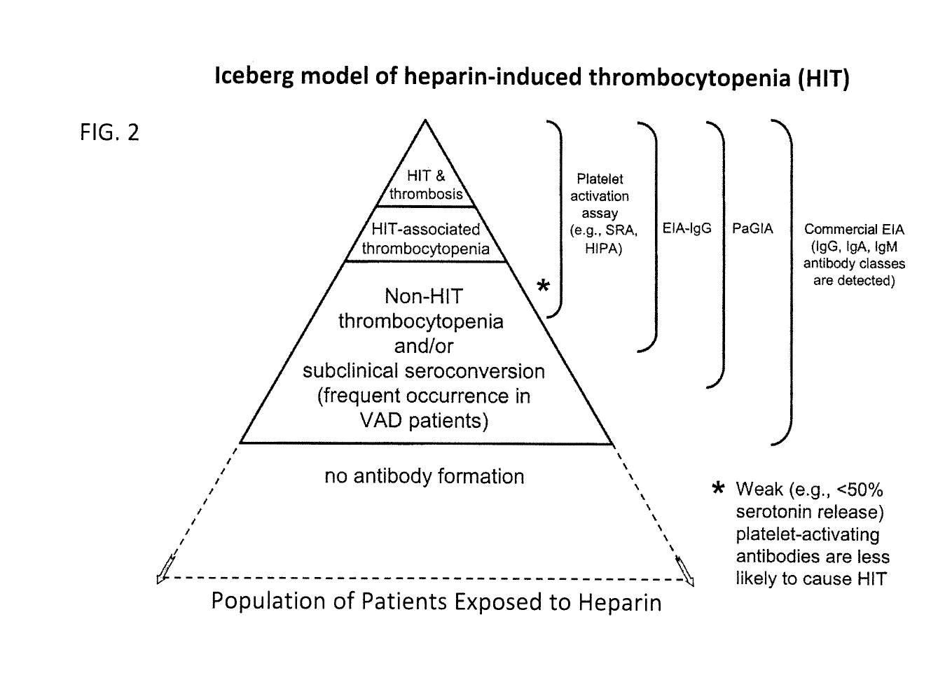

The presence of antibodies against PF4-H occurs in 3% to 60% of patients exposed to heparin. This large variation depends on the dose of heparin, the type of heparin used, the detection method, and the medical condition of the patient and the particular populations evaluated. Overall, around 5% (600,000) of patients of the 12 million patients receiving heparin every year develop heparin-induced thrombocytopenia; 20% of these patients (120,000) develop thrombosis and 30% of them (36,000) die as a result of this condition. This pyramidal distribution of patients resembles an iceberg and is regularly described as such (FIG. 2) (Warkentin et al. (2007) Ann. Thorac. Surg. 83(1):21-23). There are great differences between the sensitivity of different groups of patients to suffer HIT. For example, patients undergoing cardiopulmonary bypass surgery are more prone to develop HIT and a larger proportion of these patients end up developing autoantibodies recognizing PF4-H complexes. The incidence of autoantibodies in other patient groups is lower and seems to correlate with the severity of their pathological condition.

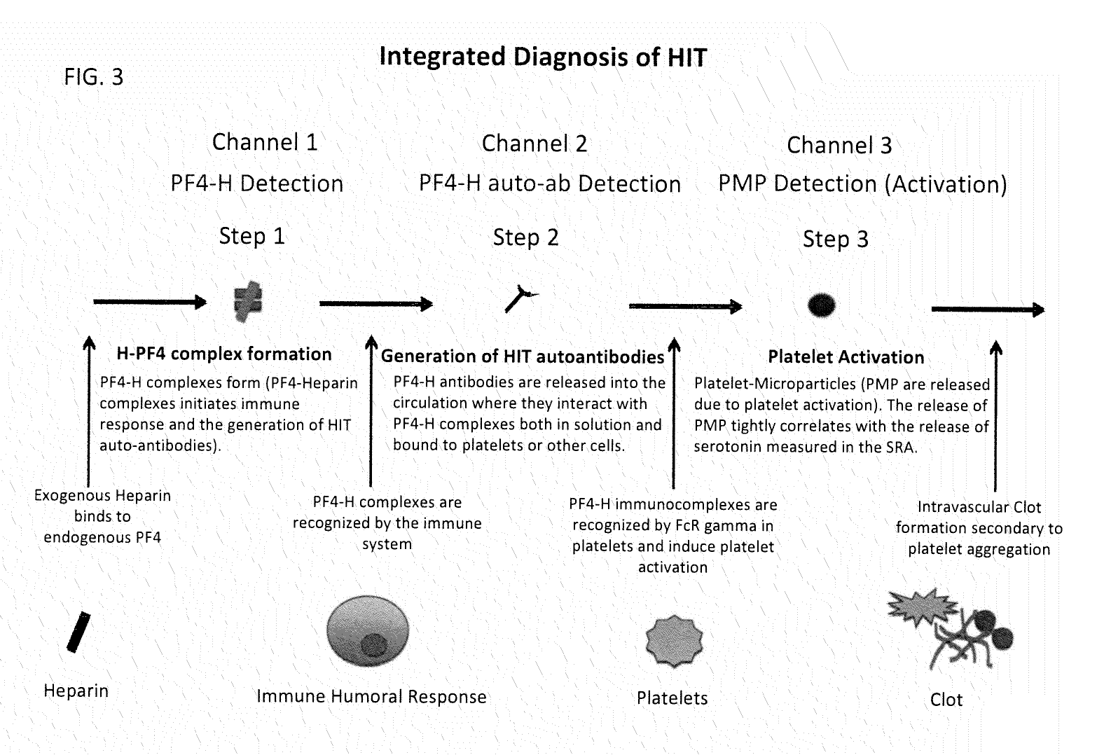

The present disclosure provides a new method for the early detection of, or predisposition to, HIT. This method provides an integrated approach for the diagnosis of HIT by incorporating quantitative information regarding the activity of the three major elements (also referred in this document as steps for being chronologically linked since they provide three different sources of information) of the pathophysiology of HIT (FIG. 3). The activity of each individual pathophysiological element is considered as a channel of information that when integrated as a whole gives the physician a much better understanding of the HIT process. This information allows the physician to take a clear therapeutic approach for each particular patient at the right time. Accordingly, this assay tracks different stages of the pathogenic process underlying HIT development and therefore provide with diagnostic information regardless of the stage of the process. The assay targets earlier diagnosis and improves the specificity of current diagnosis tests as to serve as one element in the decision-making process regarding the most appropriate therapeutic approach to undertake in each individual HIT patient. The present assay incorporates data derived from the formation of PF4-H complexes (Channel 1), the formation of autoantibodies recognizing these complexes (Channel 2), and the activation of platelets induced by the corresponding immunocomplexes (Channel 3), that result in platelet aggregation and clot formation. The assay can be formatted for lateral flow and does not require batching, and the channels or steps can be assayed concurrently or sequentially.

This assay is highly specific (e.g., reducing sensitive false positives to fewer than 10%) (no false negatives), has rapid turn around (e.g., hours), is easy to perform, and is capable of standardization. It is highly reproducible, and can run on existing laboratory equipment. It serves to predict and to identify the earlier signs of thrombotic complications related to HIT, therefore allowing early therapy to avoid the life-threatening consequences of this pathologic process. The assay also aids in the identification of patients at risk of developing HIT. Such identification serves to avoid the use of heparin, and thus prevents the development of HIT in predisposed patients. Each of the components of this assay is described below.

### Channel 1: Detection and Measurement of PF4-H Complexes

The formation of heparin-PF4 complexes leads to the development of HIT. PF4-H complexes are responsible for the activation of the immune system and the production of autoantibodies that recognize specifically PF4-H complexes. These antibodies specifically react with PF4-H complexes in circulation or associated to the plasma membrane of platelets but do not recognize the unconjugated components, heparin and PF4, individually. The formation of these complexes is dependent on the relative concentrations of heparin and PF4, the presence of interfering compounds that accelerate or delay their formation, as well as their association to cellular surfaces (including endothelial cells, platelets, microparticles, red blood cells, white blood cells and other cellular components).

As used herein, the term “microparticle” refers to cellular derived, membrane-associated multimolecular complexes or structures released by a process of cellular activation (e.g., by platelet activation) and/pr a passive process of cellular disintegration (e.g., apoptosis).

Association to exposed collagen and other extracellular matrix components as well as other microenvironmental factors also contribute to the tendency of these molecules to associate. Presentation of these complexes to the immune system results in the production of antibodies that recognize new epitopes created by the interaction of PF4 and heparin but absent in the native PF4 and heparin molecules. Inflammation and stimulation due to other pathologic agents can enhance this process by increasing the expression and secretion of PF4 into circulation, and by structurally modifying its three dimensional structure.

Inflammation and severe stress induce the release of PF4 into circulation. This is one of the reasons patients affected with cardiovascular disease or undergoing other stressful or complicated surgeries are more susceptible to HIT. Although, PF4 can be directly measured, postranslational modifications induced by stress as well as other local conditions can affect its affinity to form complexes with heparin. For this reason, the present assay specifically measures the complex between heparin and PF4 (PF4-H) and not free circulating PF4. Although measurement of the concentration of PF4-H complexes by itself does not identify patients suffering from HIT, the concentration of PF4-H is an essential component of the pathophysiology of HIT and therefore it serves as an indicator of the relative risk that a particular patient has to develop the disease. Three different approaches are described herein to quantify PF4-H complexes.

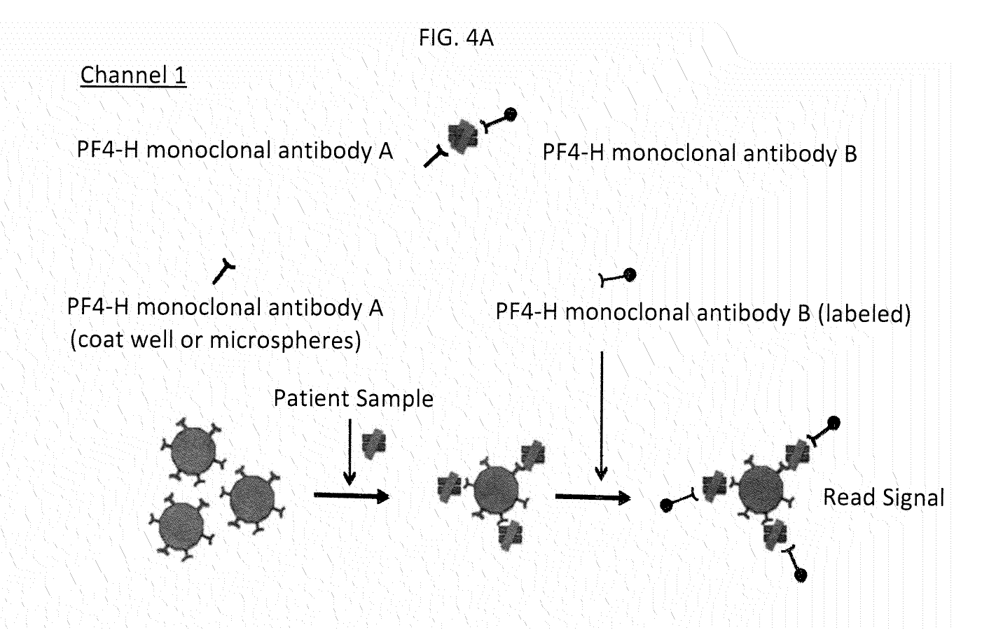

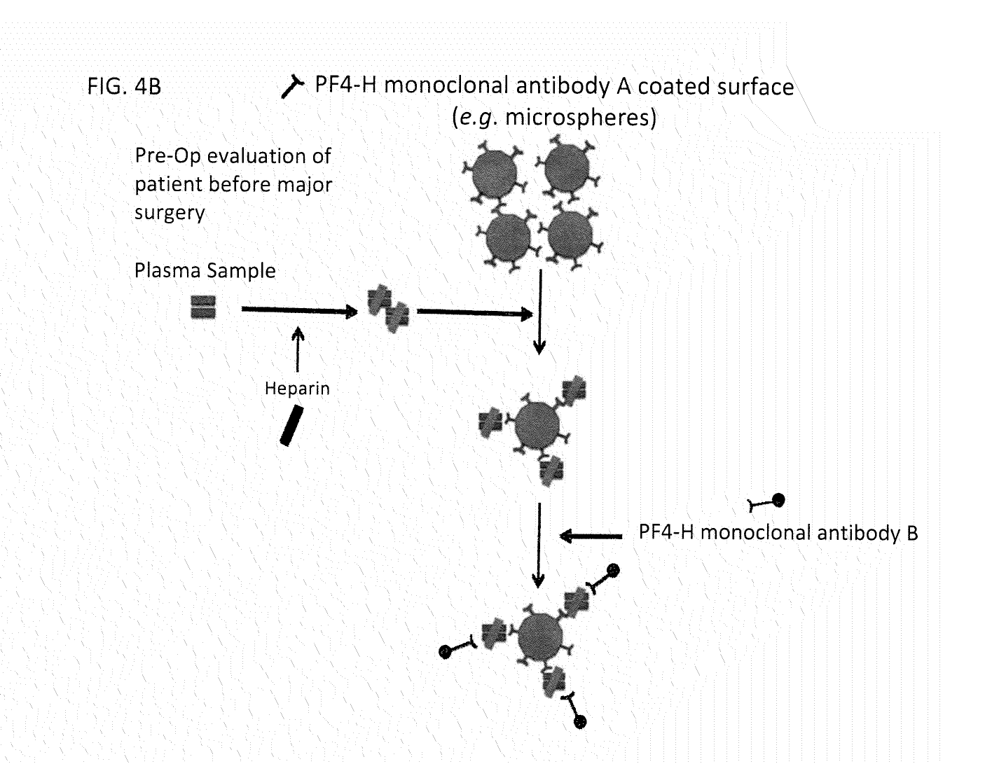

The present assay utilizes a pair of monoclonal antibodies or antibody binding fragments that specifically recognize the PF4-H complex but do not recognize original components in (heparin and PF4) in isolation (FIG. 4A). Binding fragments of monoclonal antibodies are known (Fv, Fab, F(ab)₂, etc).

The monoclonal antibodies can be made by methods well known in the art (see Suvarna et al. (2007) Blood, 110:4253-60). Likewise, antibody-binding fragments can be made according to methods well known in the art.

This pair of antibodies (HPF4 mAb A and HPF4 mAb B) or binding fragments thereof, recognizes different epitopes within the complex and therefore can be used in an immunoassay, such as a sandwich immunoassay (e.g., ELISA, ELISPOT, xMAP multiplexing and others). The assay is performed in plasma or serum to determine the concentration of free circulating PF4-H complexes. To evaluate the concentration of PF4-H associated to platelets and microparticles, plasma rich in platelets and microparticles is prepared by low speed centrifugation and then treated with a low stringency detergent such as Triton X100. The resulting sample is used to measure the total concentration of PF4-H. By calculating both the concentration of PF4-H complexes in solution and after Triton treatment we calculate the difference that represents the fraction associated with the membrane compartment of platelets and microparticles (FIG. 4A). Other non-ionic detergents such as Brij-98, Nonidet P40 (NP40) as well as non-detergent methods such as the use of alkaline carbonate (0.5 M Na₂CO₃) can also be used for lipid raft preparation.

The measured concentration of PF4-H correlates with the appearance and severity of HIT. A concentration threshold, representing a significant risk, is created using a homogeneous population of patients. This information can be interpreted individually or integrated in the context of data provided by other channels of the ID-HIT assay as well as in the context of clinical findings observed in each individual patient.

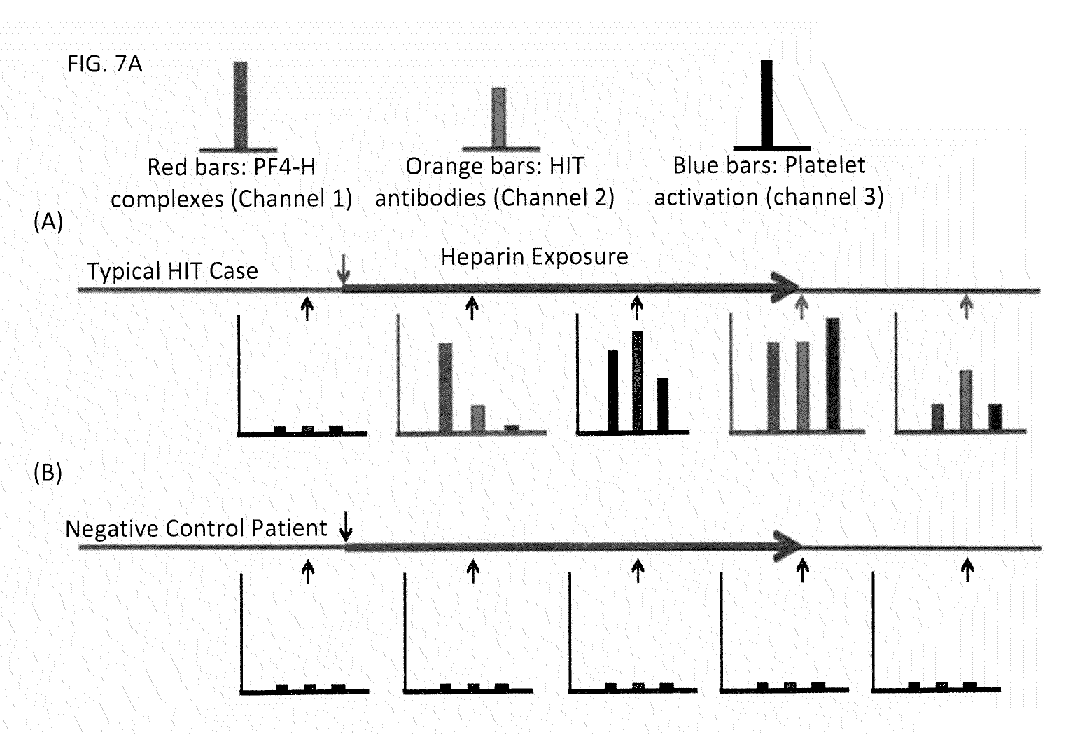

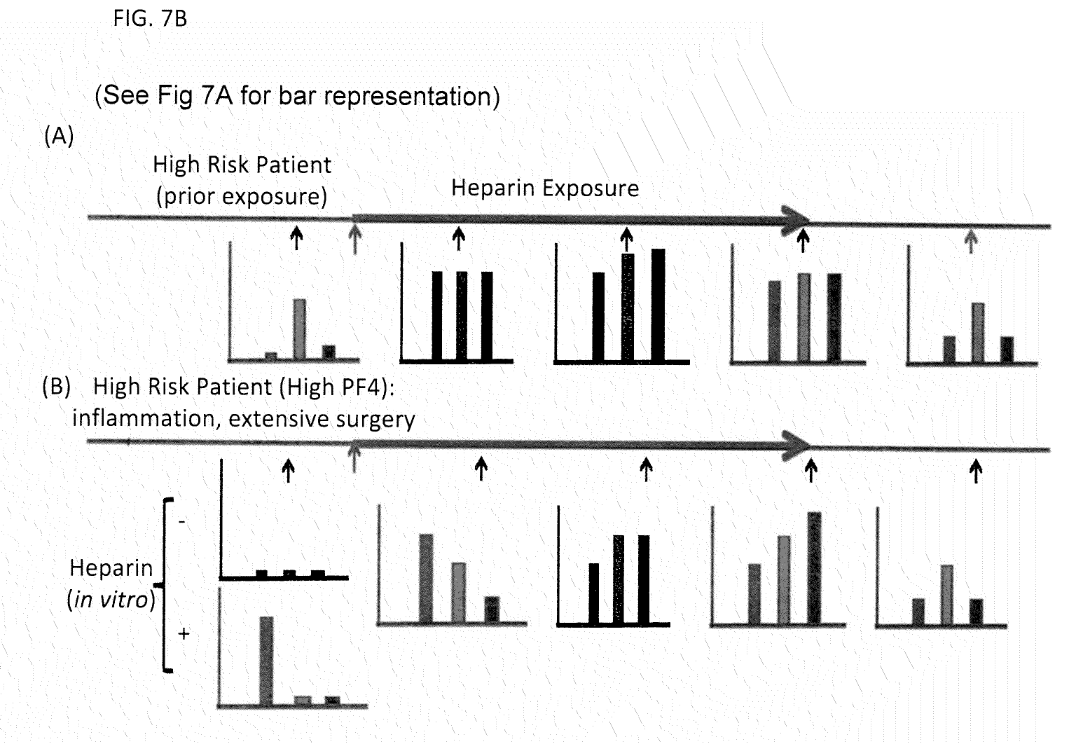

Channel 1 of the present assay can also be performed as part of a pre-op evaluation of a patient before major surgery or heparin treatment. In this case, exogenous heparin (0.1 i.u./ml-10 i.u./ml) is added to the sample in vitro prior to running the assay (FIG. 4B). The quantified PF4-H complexes represent an indication of the propensity of the patient plasma to form these complexes if heparin therapy is initiated. This ability is influence by the concentration of circulating PF4 and its affinity for heparin as well as by other local factors difficult to measure individually. PF4-H complex concentration is measured with immunodetection methods such as sandwich ELISA, Luminex xMAP, etc. The concentration of PF4-H complexes formed in these conditions provides information concerning the concentration, availability, and propensity of PF4 to associate with heparin and therefore inform us of the predisposition of a specific patient to develop HIT. A relative risk value is assigned to each concentration depending on empirical values obtained within the patient group population. FIG. 7B describes the characteristics of a patient with a high predisposition to develop HIT due to a high level of PF4-H complex formation. When the patient sample is analyzed prior to the start of heparin treatment, a high level of PF4-H complexes is detected in the presence of in vitro added heparin. Several factors may contribute to complex formation including a high concentration or PF4 secondary to inflammation, the presence of posttranslational modifications, as well as metabolic or toxic alterations that support or enhance the formation of the PF4-H complexes. These patients are at high risk to develop HIT autoantibodies and platelet activation at a later time if heparin therapy were to be initiated (FIGS. 4A and 4B).

The formation of the PF4-H complexes is facilitated when the components involved are associated with a solid surface such as a cell membrane. Inflammation induces the release of circulating microparticles into the circulation. This effect is proportional to the intensity of the inflammatory stimulation and therefore is higher in certain groups of patients such as those undergoing complicated stressful surgical procedures. The release of microparticles in these conditions serves to amplify the inflammatory process, as well as to support the activation of pro-thrombin and clot formation. It also increases the surface area on which PF4-H can be assembled. Therefore, the measurement of the concentration of PF4-H complexes associated to MPs can be used as an indicator of propensity of a particular patient to develop HIT. To take into account these membrane-associated complexes, the samples containing platelets and microparticles are treated as follows: The blood sample (e.g., 1 ml) is centrifuged at low speed (e.g., 1500 rpm) for 15 minutes. The upper fraction containing plasma rich in platelets and microparticles is collected and pretreated with low ionic strength detergent (e.g., Triton x 100; 1%) for 30 seconds on ice. This sample now containing plasma and lipid rafts complexes derived from platelets and microparticles, is mixed and incubated according to one step of the assay (Channel 1).

### Channel 2: Detection and Measurement of Autoantibodies Specific For PF4-H Complexes

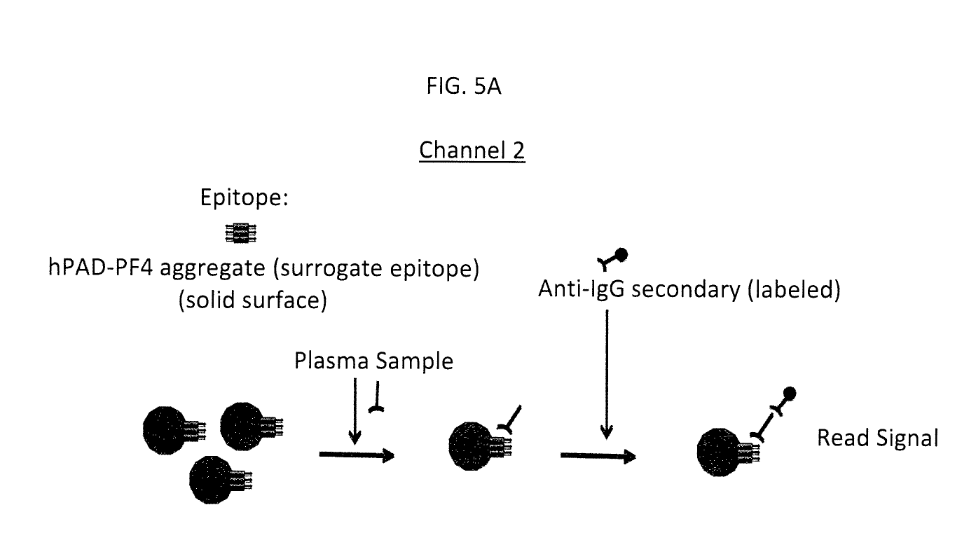

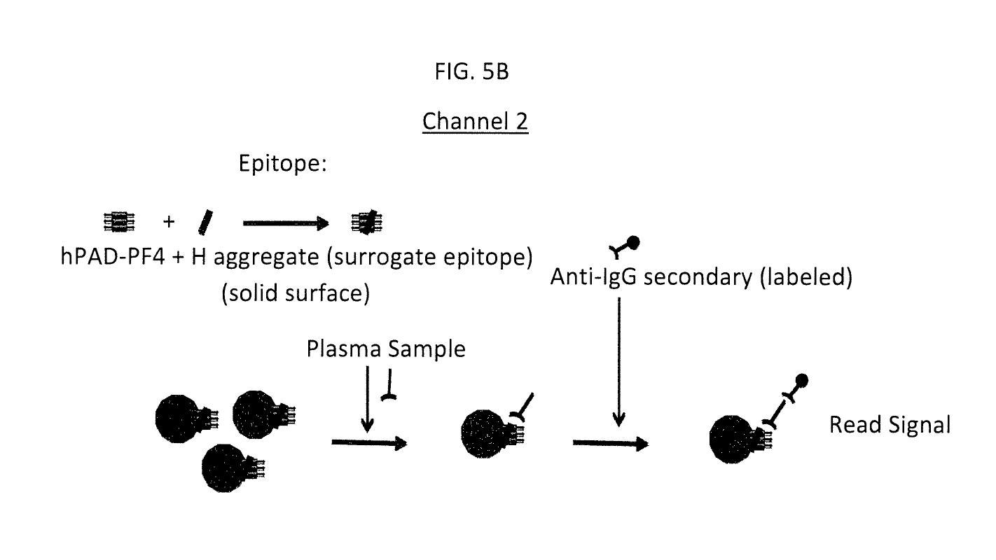

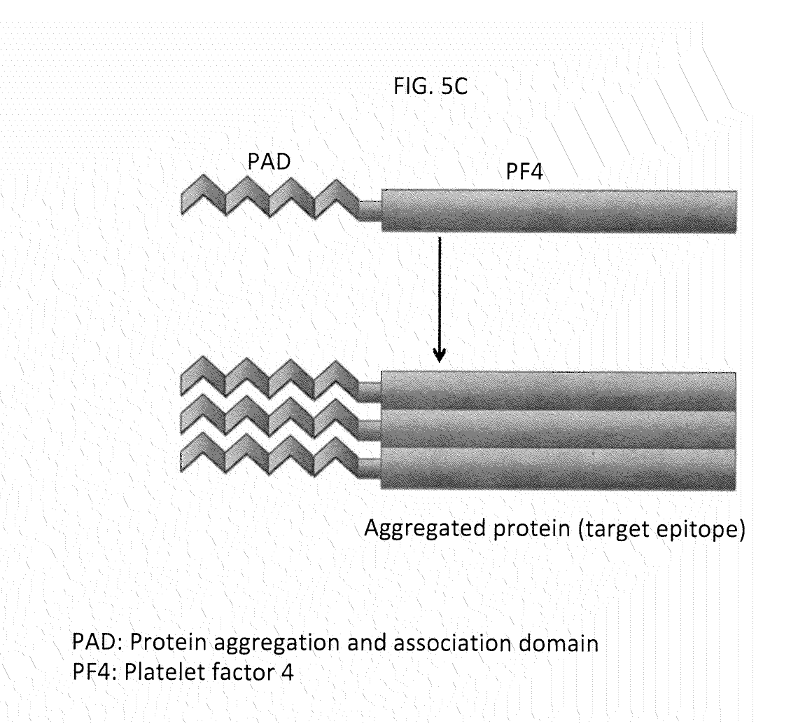

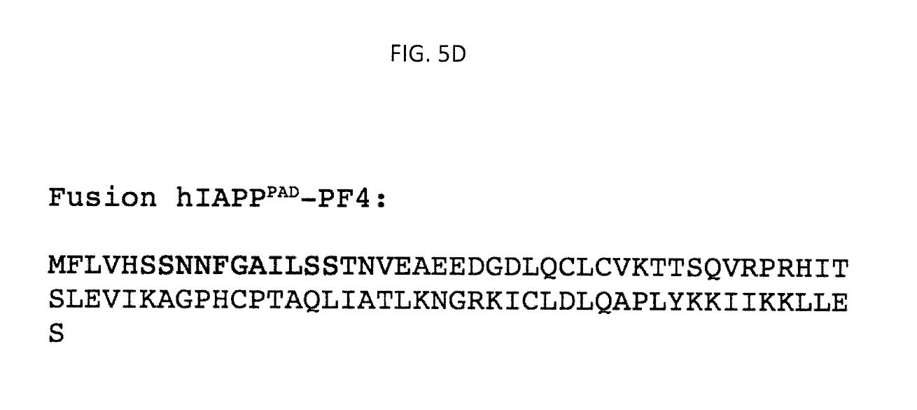

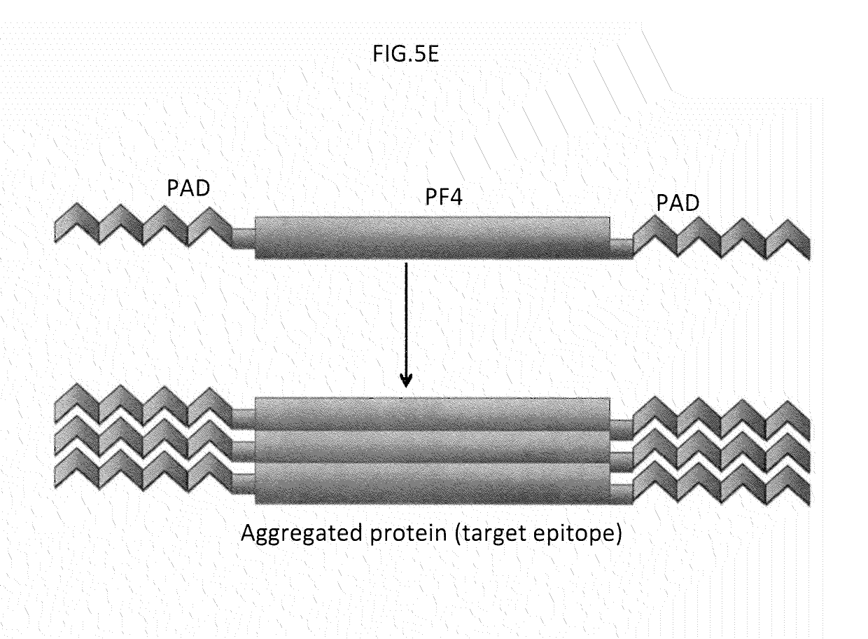

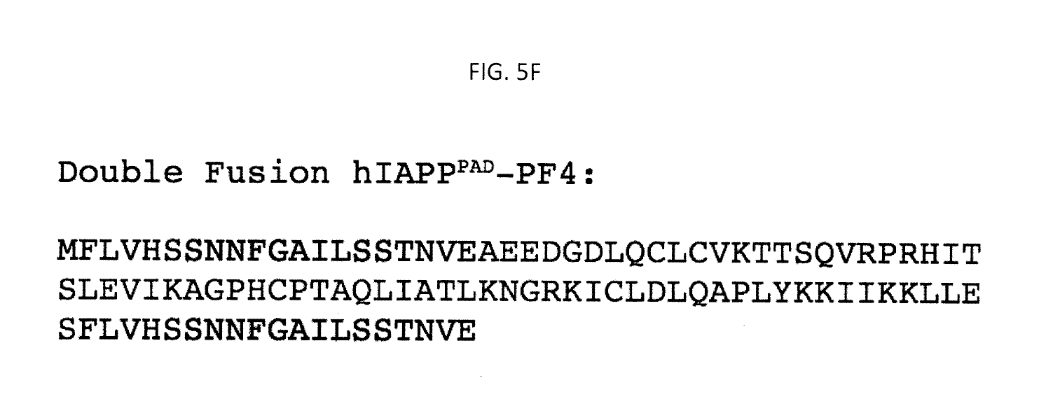

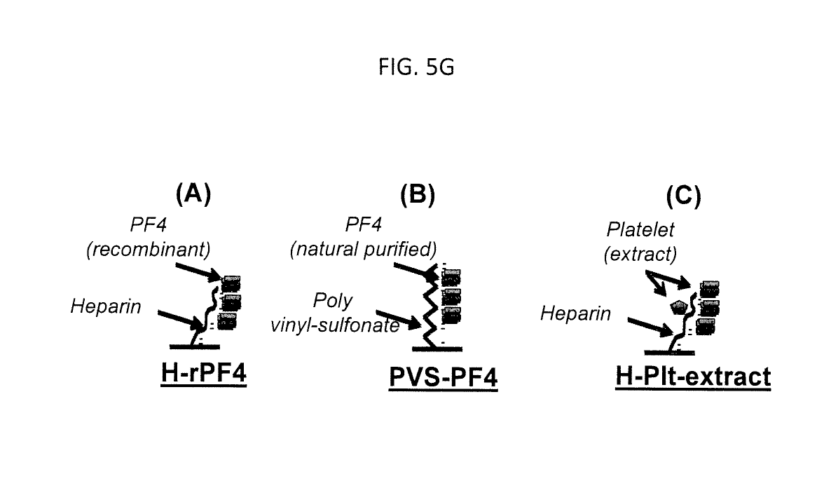

Existing methods for the detection of PF4-H autoantibodies primarily rely on immunoassays to detect the presence of antibodies specific for the heparin-PF4-H complexes or for surrogate antigenic determinants previously immobilized on a solid surface (FIG. 5G). These immunoassays also use a secondary enzyme-labeled antibody that recognize the Fc region of the HIT antibodies present in each patient. Different secondary antibodies with specificity toward the constant domain of the HIT antibodies allows for the specific recognition of the type of immunoglobulin present in each patient (IgG, IgA or IgM). These assays differ in the way the antigenic PF4-H or surrogate target is created. One approach uses heparin and recombinant PF4 bound to a solid surface, another substitutes heparin for a negatively charged polymer: polyvinyl-sulfonate that, combined with natural purified PF4, creates an equivalent target for HIT antibodies, and a third assay uses heparin bound to a solid surface that has been exposed to an extract from platelets in order to create a more natural antigenic complex (see FIG. 5G). The role of heparin is to facilitate the formation of larger PF4 aggregates through neutralization of positive charges on the PF4 molecule. These multi-molecular aggregates of PF4 serve as the real antigenic determinant in the pathogenesis of HIT.

The new surrogate epitope described herein is created by incorporating in the PF4 protein one or more domains with self-aggregation capabilities. These domains substitute for the function of heparin and or poly vinyl-sulfonate by forcing PF4 to aggregate and to form antigenic determinants equivalent to the ones presented to the immune system in HIT. The sequence of these protein aggregation domains (“PADs”) corresponds to segments of the sequence of other proteins with such aggregation capability. Useful domains include the leucine-zipper domain of CHOP (Genbank Acc. No. GC12M054948) and C/EBPβ (Genebank Acc. No. GC20P045555), the amylin aggregation domain (Genebank Acc. No. GC12P021299), and the hydrophobic repeat domain of the heat shock transcription factor (Genebank Acc. No. GC16P069593). These amino acid sequences contain structural hydrophobic amino acids exposed on their surface that allow for a strong protein-protein interaction that results in a stable aggregation of the proteins involved. By creating a fusion protein containing one (FIG. 5C) or more (e.g., FIG. 5E) of these PAD domains and PF4, more homogeneous and more stable PF4 aggregates can be created than those created by heparin or polyvinylsulfonate in the assays. The use of these aggregates in the detection of the pathogenic autoantibodies responsible for HIT improves the performance of current available assays based on the technologies described above.

The use of this approach is demonstrated in two examples in FIG. 5C-5F. A recombinant expression vector encoding or PAD-PF4 or PADₙ-PF4 contructs is generated in vitro by fusing the corresponding DNA coding sequences (e.g., FIG. 5C and FIG. 5D) within an appropriate plasmid. A number of protein expression vectors are currently available to use in both prokaryotic (e.g. bacteria) and eukaryotic (e.g. yeast or mammalian). An example of these vectors includes the PGEX, and His-tag vectors that can be commercially obtained from GE Healthcare (Piscataway, N.J.) and Invitrogen (Carlsbad, Calif.). The vectors enter bacteria, yeast, or other eukaryotic or prokaryotic cells and instruct the production of the protein they encode. The encoded fusion protein in this case is then expressed and purified by well-known standard molecular biology technologies. The purified fusion protein is then adhered to a solid surface such as, but not limited to, the bottom of a microwell plate or microbeads such as polystyrene microspheres. Adherence is enabled by direct absorption to surface and or facilitated by other current available standard technologies. These bound aggregates are incubated in the presence of a sample from a patient suspected of suffering from HIT, and the bound antibodies detected using an anti-human immunoglobulin antibody (e.g., anti-IgG, anti-IgA, anti-IgM) appropriable labeled (FIG. 5A). Useful patient samples include fluid samples such as, but not limited to, blood, plasma, serum, saliva, urine, lacrimal excretions, mucous, vaginal secretions, and seminal fluid.

In another embodiment of this approach, un-fractionated heparin is added during the process of PAD-PF4 aggregation in order to increase the rate of epitope formation (FIG. 5B). Labels useful for marking and detecting the anti-human immunoglobulin utilized for the recognition of the HIT antibodies associated with the solid surface include, but are not limited to, commercially available enzymes (e.g., peroxidase, alkaline phosphatase, luciferase)), fluorescent compound (e.g. biotin, avidin, fluoresceine, rhodamine, Cy3, Cy2, Cy5), and radioactive labels (e.g. ¹³¹I, ³H, ¹⁴C, ³²P). Quantification analysis of the reading output signal provides information concerning the presence of HIT pathogenic antibodies in a particular patient. Interpretation of the output data can be performed in isolation or in the context of additional data provide by the present ID-HIT assay.

### Channel 3: Evaluation and Quantification of Platelet Activation Secondary to HIT

Thrombosis, the most severe complication of HIT, is induced by the binding of HIT immunocomplexes (PF4-H complex and HIT antibodies) to the platelet membrane FcγIIa receptors inducing platelet activation. Platelet activation results in the release of pro-coagulant factors such as serotonin, and in the formation and release of microparticles. Microparticles are vesicles released by platelets surrounded by a membrane derived of the platelet plasma membrane. Thus, microparticles contain both membrane-associated proteins as well as intracellular cytoplasmic content derived from the original cell. In HIT, microparticles are released to amplify the function of platelets by facilitating clot formation. They serve as solid phospholipid substrate (increased surface area) on which a prothrombin-activating complex ensembles. They also contribute to clot formation by interacting with the vascular endothelium and other intravascular components including other platelets. In HIT, microparticles are released early due to initial platelet activation secondary to the presence of circulating H-PF4-IgG immuno-complexes that interact with the Fc receptor in the platelets. In addition, the sera from HIT patients can induce the formation of microparticles when exposed to previously isolated-washed platelets. Detection of platelet microparticles by flow cytometry correlates (96%) with platelet activation measured.

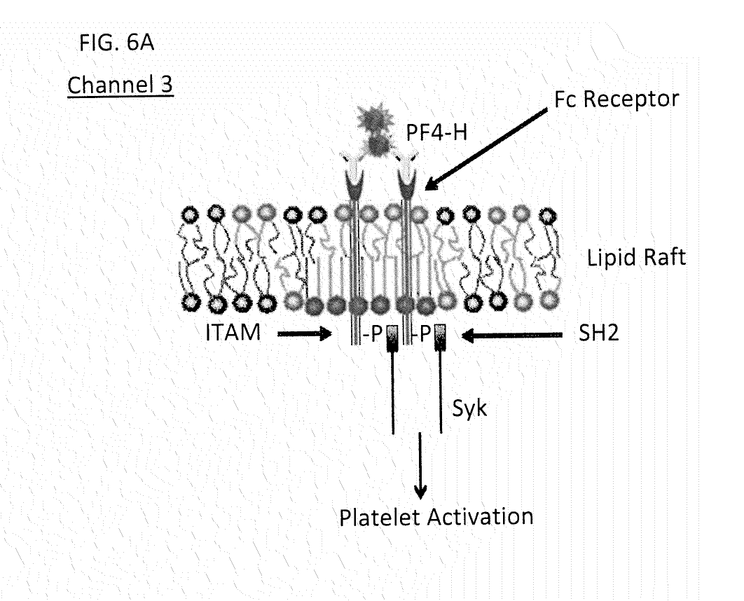

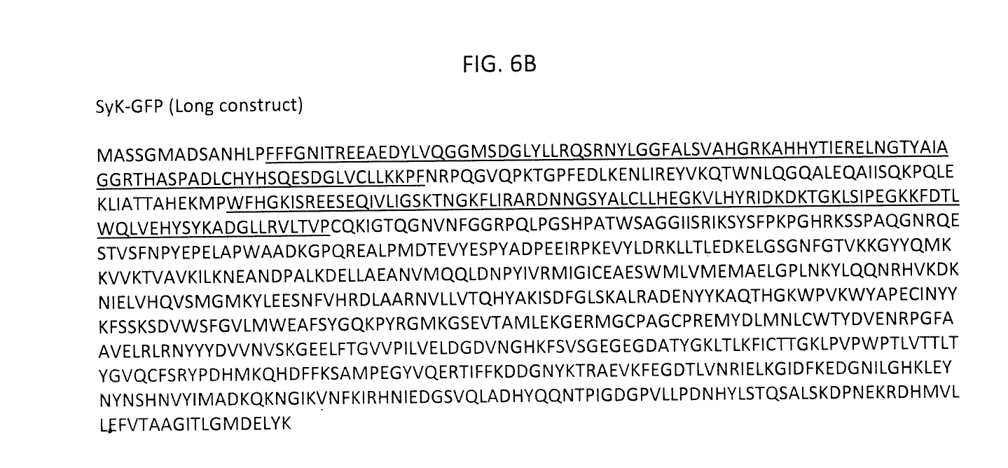

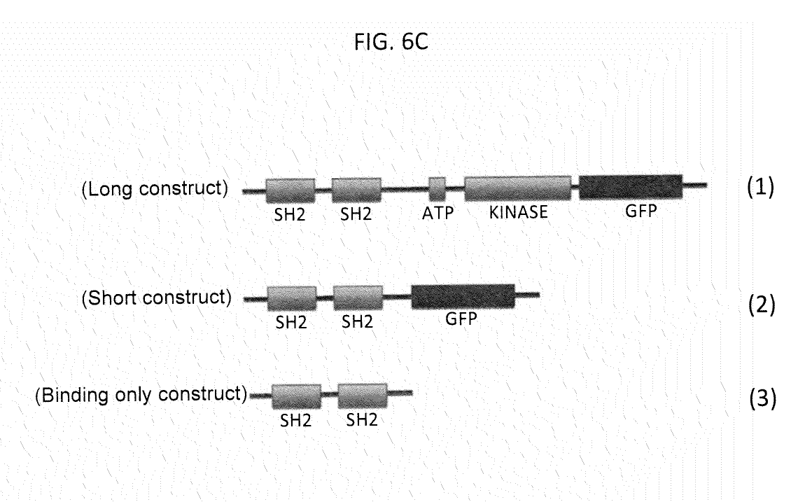

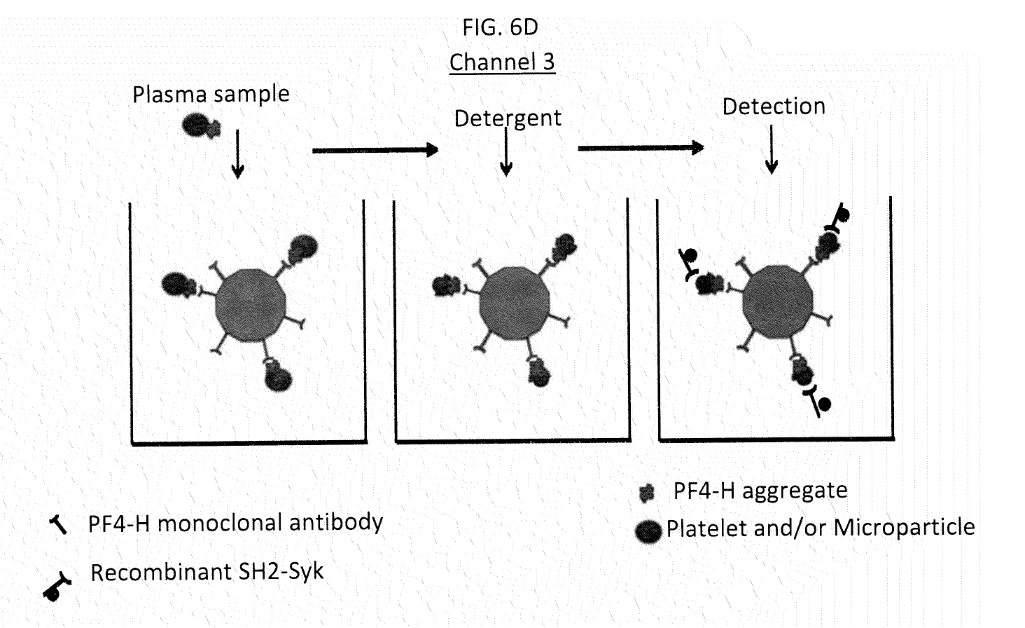

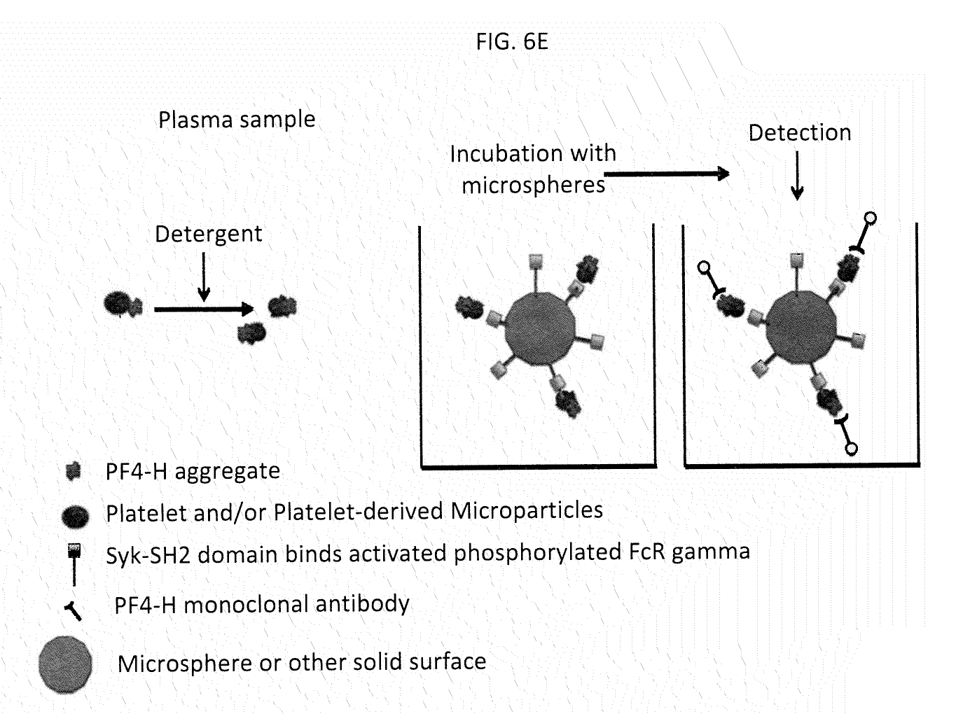

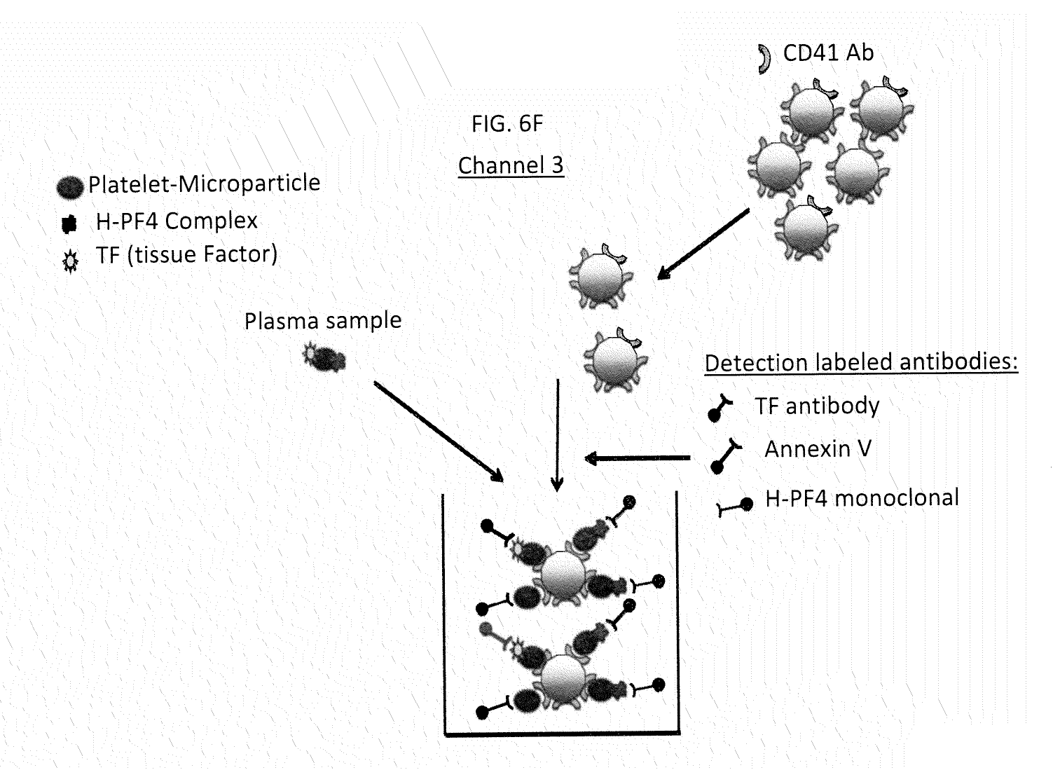

In Channel 3 of the present assay, the level of platelet activation induced by HIT is measured by evaluating the level and activity of microparticles derived from activated platelets, as well as by detecting and quantifying the activation state of Fc receptors localized within the membrane of platelets and microparticles. Activated FcR receptors reside within lipid rafts formed by the interaction of HIT immunocomplexes with the membrane of platelets (FIG. 6A). Three representative but non-limiting methods are described herein to evaluate the presence of platelet activation associated with HIT.

Complexes containing PF4 and heparin can be found both free in circulation and in association with the platelet and microparticle membranes. At least a fraction of the complexes associated with membranes corresponds with the Fc receptor (FcRgIIa) associated fraction responsible for thrombotic complications (FIG. 6A). In one embodiment, the present assay uses a monoclonal antibody recognizing the complex PF4-H, attached to a solid surface to isolate microparticles present in the patient sample (FIG. 6D). Once the microparticles are bound, the plasma membrane is disrupted (e.g., with a detergent solution, i.e., Triton X100) or mechanical disruption, to expose the internal side of the microparticle while preserving the lipid raft domains. These patch or raft domains associate with the immobilized monoclonal PF4-H antibodies and contain functionally active Fc receptors. A second labeled reagent is used to recognize the phosphorylated internal domain of the activated Fc receptors (though their ITAM domains). This reagent comprises the SH2 domain of the protein kinase SyK associated with a label such as green fluorescent protein (GFP) (FIG. 6B). FIG. 7 shows the functional protein domains of the SH2-SyK construct that uses GFP as the label. Both full length (long construct) and a short version incorporating only the amino-terminal region of SyK, responsible for binding to activated-FcR, are depicted. This short amino-terminal region, containing the two SH2 binding domains, is also produced as a recombinant protein or peptide (Binding-only construct) to serve as the specific-binding site for lipid rafts derived from microparticles and platelets. These rafts contain the activated Fc receptors exposing the phosphorylated ITAM domains. The sequence of the SyK-GFP protein construct is shown in FIG. 6D.

Other labels such as enzymes (e.g. peroxidase, alkaline phosphatase, luciferase) chemically active compounds (e.g. biotin, avidin, fluoresceine, rhodamine, Cy2, Cy3, Cy5), and radioactive compounds (e.g. ¹³¹I, ³H, ¹⁴C, ³²P), can also be used depending on the requirements of the assay platform used. Smaller fragment of SyK containing at least the SH2 binding domains can also be used. For example, in another embodiment (FIG. 6E), the SyK sequence containing the interacting SH2 domains but not the full-length sequence is used. This domain is conjugated on the surface of a solid support (e.g., microspheres, beads, or microwells) in order to capture the activated Fc-R present in lipid rafts of platelets and/or microparticles. These lipid rafts are generated by pre-treating the sample using standard protocols (e.g. treating the sample with Triton-X100 at 4° C.).

Another approach to Channel 3 of the assay evaluates the level of platelet activation associated with HIT by detecting the presence and activity of platelet-derived microparticles in plasma (FIG. 6F). This embodiment utilizes antibodies recognizing the platelet-specific CD41 (GP-IIb integrin) membrane marker, attached to a solid surface. Once microparticles are immobilized, antibodies against PF4-H and tissue factor (TF) as well as phosphatidyl serine antibodies or annexin V are used to evaluate the thrombogenic potential of the associated microparticles. This approach utilizes centrifugation at a force and time sufficient to separate microparticles from other cellular components in the blood sample. This can be achieved in one centrifugation step (high speed) or two centrifugation steps (low speed+high speed) using the settings as follows: The blood sample (e.g., 1 ml) is centrifuged at low speed (e.g., 1,500 rpm) for 15 minutes. The upper fraction containing plasma rich in platelets and microparticles is collected and centrifuged at a higher speed (e.g., 12,000 rpm) for 5 minutes. The upper phase containing plasma rich in microparticles (but no platelets) is collected and incubated as described in Channel 1. The sample contains intact microparticles that can be immobilized with antibodies recognizing membrane markers. Its inner composition is analyzed after membrane disruption with Triton x 100.

### Interpretation and Treatment

Quantitative data from the present assay allows the physician to determine the need to avoid heparin use in those patients with the highest probability of suffering HIT. In these patients a direct thrombin inhibitor (such as Argatroban and Lepidurin) would be the most appropriate agent to avoid the development of thrombotic complications during their hospital stay. Repeated ID-HIT testing at regular intervals during heparin administration allows the physician to detect the early signs of HIT development and to determine the most appropriate time to discontinue heparin treatment and to substitute an alternative anticoagulant agent.

Use of the ID-HIT assay also identifies certain patients predisposed to develop HIT even after heparin administration has been discontinued. Accordingly, the indications for ID-HIT are multiple: a) screening of patients before onset of heparin administration; b) monitoring of patients undergoing heparin administration; and c) screening for Delayed-Onset HIT. Table 1 describes what patients would benefit from ID-HIT testing and how.

TABLE 1
Appropriate Utilization of ID-HIT ™ Testing

| Time when Indicated | Previous Heparin Use | No Previous Heparin Use |
|---|---|---|
| Prior to heparin use | Yes (modified test | Yes (modified test |
| (as part of Pre-Op | using exogenous | using exogenous |
| testing) | heparin) | heparin) |
| Day 2 after initiation | Yes | No |
| of heparin treatment | | |
| Day 5 after initiation | Yes | Yes |
| of heparin treatment | | |
| Day 8 after initiation | Yes | Yes |
| of heparin treatment | | |
| Day 11 after initiation | Yes | Yes |
| of heparin treatment | | |
| 5 days after | Yes, if low but rising | Yes, if low but rising |
| discontinuation | levels of HIT c or | levels of HIT c or |
| of heparin | HIT ab | HIT ab |

Thus, it is recommended that all patients requiring heparin anticoagulation should be tested with ID-HIT in order to allow for early detection of HIT and the prevention of the severe thrombotic complications.

FIG. 7A shows exemplary graphic representations of the results from Channels 1, 2, and 3 testing of a sample from a typical HIT case before, during, and after heparin administration (A), relative to an untreated control patient sample (B). In contrast, FIG. 7B shows graphic representations of the results from Channels 1, 2, and 3 testing of a sample from a high risk patient having prior exposure to heparin before and after secondary heparin administration (A), and from a high risk patient having experienced extensive surgery or inflammation in which heparin is added to the sample (B). The concentration of PF4-H complexes is represented by red bars in FIG. 7A, the concentration of HIT antibodies as orange bars, and the level of Fc receptor activation in platelet derived lipid rafts, reflecting platelet activation, is represented by blue bars.

As shown in FIGS. 7A and 7B, and according to Table 1, the assay is performed at different times during therapy (e.g., before heparin initiation, 2 days after heparin administration, 5 days after, 8 days after, and 11 days after or until heparin therapy is not needed. ID-HIT is also indicated after discontinuation of heparin therapy in order to evaluate the possibility of development of the Delayed-Onset form of HIT at least in suspected patients (Table 1). When the test is performed before heparin treatment is started, exogenous heparin is added in order to allow for PF4-H complexes to form in vitro) (FIG. 7B). Testing at day 2 is performed for patients with prior exposure to heparin in order to detect early initiation forms of HIT that characterize this group of patients. Testing after completion of heparin therapy is also performed in those patients with low but rising levels of HIT antibodies and or HITc complexes in order to prevent the development of late thrombotic complications associated with heparin use (late onset HIT).

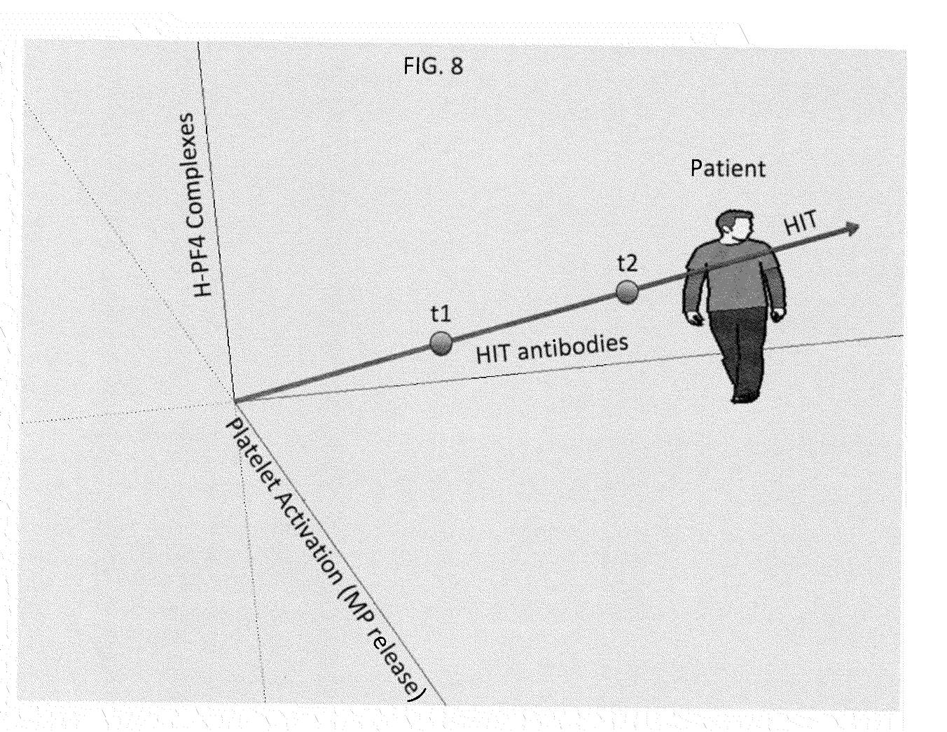

Quantitative information derived from ID-HIT closely represents a clear pathologic stage of the disease; two or more tests distributed over time serve to project the progression of the pathological process over the near future (see FIG. 8). This represents valuable information for the clinician on which to support a clear therapeutic approach that is not possible today with the current available tests in the market and their limitations.

The following examples are intended to further illustrate certain embodiments of the invention and are not limiting in nature.

### EXAMPLES

### Example 1

Simultaneous Processing Protocol

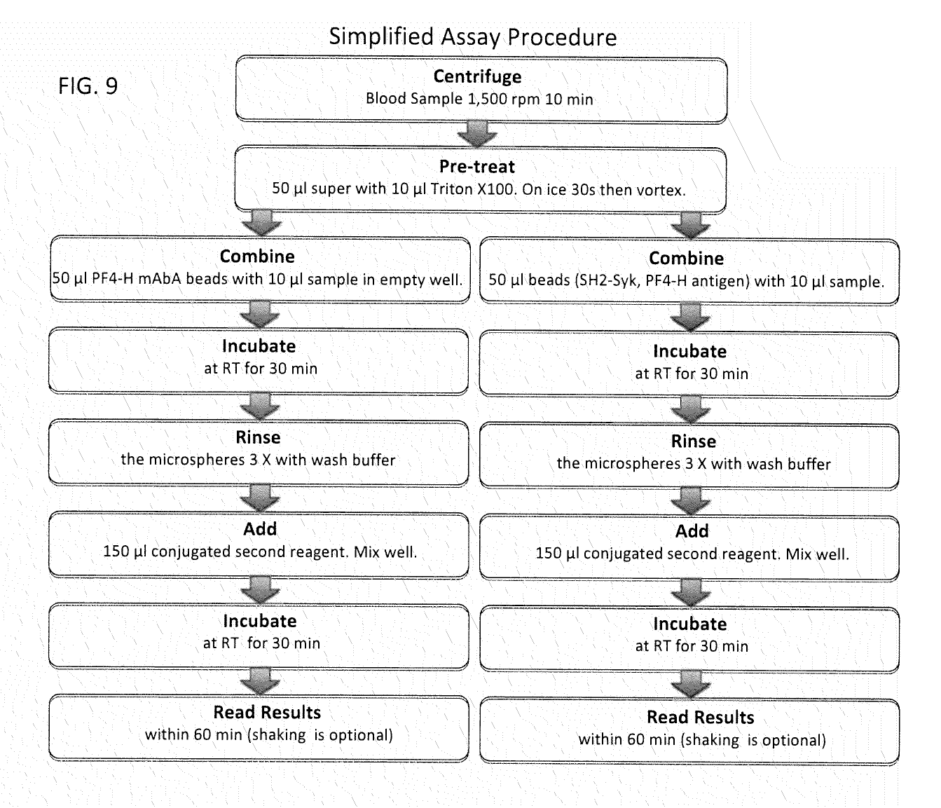

ID HIT is run in a Luminex xMAP or similar platform, although it can also be run as three separate standard immunoassays (e.g., ELISAs). Depending on which platform is used, the approximate total time required for the completion of the assay ranges from 2 hours to 3 hours. This protocol is delineated in FIG. 9.

Blood is collected (1.8 ml) and anti-coagulated with sodium citrate (3.2%). Standard blue cap and lavender cap vacutainers (BD Bioscience, Franklin Lakes, N.J.) are used to facilitate blood collection and processing.

The sample is centrifuged at 1,500 rpm for 10 min to obtain plasma rich in platelets (PLT) and platelet-derived microparticles (PMP). The supernatant containing platelets and microparticles is then used for the analysis. A 0.4 ml aliquot of the supernatant is used to run the entire ID-HIT assay and the remaining sample can be used to repeat the assay if needed.

The sample is prepared for analysis by treatment with Triton-X100 (50 μl of a 10% solution to achieve a final concentration of 1%) at 4° C. for 10 min to dissolve the plasma membrane of platelets and microparticles exposing the intracellular surface of lipid rafts and releasing PF4 complexes. This treatment is performed in lipid raft compatible buffer (e.g. 50 mM MES, 100 mM NaCl and pH 7.4) with protein phosphatase inhibitors (e.g., 100 mM NaF; and 100 mM Na₃VO₄) in order to prevent dephosphorylation of the activated Fc receptor ITAM domains and protease inhibitors (e.g. 1 μM leupeptin, 0.3 μM aprotinin, and 1 mM EDTA). This step also releases PF4-H complexes and antibodies associated with the platelets and platelet-derived microparticles, and makes them available for testing.

An aliquot of 100 μl pretreated sample is then incubated for 30 min with polystyrene microspheres (500 μl suspension from Luminex Corp., Austin, Tex.) These microspheres are individually identified using an internal fluorescent signature introduced during the manufacturing process. Specific label information is provided by the manufacturer (e.g., Luminex Corp., Austin, Tex.). Exposed on their surface are three different reagents: (1) a monoclonal (PF4H mAb A) that recognizes PF4-H complexes; (2) an aggregate of PF4-PAD that serves as antigen for HIT antibodies; and (3) a peptide containing the SH2 domain of SyK kinase that specifically recognizes the intracellular ITM domain of activated Fc receptors.

The suspension is then rinsed three times with 300 μA phosphate buffer saline (0.15M PBS, pH 7.2) to eliminate the excess of reagents and reactants after first incubation.

A second incubation is performed with fluorescent-labeled PF4H monoclonal antibodies (mAb-A and mAb-B) and anti-IgG monoclonal antibodies (anti IgG) for an additional 30 min period. Reading and quantification is then performed within 1 hr using a Luminex MAGPIX, Luminex 100/200, or equivalent instrument.

### Example 2

### Sequential Processing Protocol

The procedure for ID HIT can easily be divided into three different assays that can be run as three independent ELISA assays in those laboratories where the equipment required to perform multiplex assays is not available. These can be set up sequentially by one technician or even in parallel if laboratory automation is available.

Blood collection, centrifugation and sample pretreatment are identical to the ones described above for the simultaneous test protocol.

In this sequential protocol however, the capture reagents (a.—antibody A against PF4-H complexes, b.—the antigenic determinant (PAD-PF4) for HIT antibodies detection, and c.—the peptide containing the two SH2 domains of SyK) are absorbed onto the bottom surface of microwell plates.

The procedure involves rinsing of the microwell plates with phosphate buffer saline (PBS) at room temperature (RT) in order to reconstitute and equilibrate the attached reagents. 50 μl of the processed sample is added into the microwells and incubated for 30 min at 37° C. The contents are decanted and washed three times with 300 μA PBS buffers. 50 μA of labeled conjugated secondary antibody is then added (a. antibody B against PF4-H complexes; b. anti-IgG for HIT antibodies detection; and c. antibody A/B against PF4-H complexes for channel-3). The concentration of these antibodies is individually optimized but ranges between about 1:20,000 to about 1:30,000 dilution. These antibodies have previously being conjugated with alkaline phosphatase, and incubated at 37° C. The sample is rinsed 3 times with 300 μl of PBS buffer, decanted or aspirated before adding 100 μA of alkaline phosphatase PNPP (p-nitrophenyl phosphate) substrate, and incubated at RT for 30 min in the dark. The reaction is stopped by adding 100 μA of stop solution (3M NaOH). The absorbance (OD) at 405 or 410 of each well is read within 1 hr using a reference filter of 490 nm.

### EQUIVALENTS

Those skilled in the art will recognize, or be able to ascertain, using no more than routine experimentation, numerous equivalents to the specific composition and procedures described herein. Such equivalents are considered to be within the scope of this invention, and are covered by the following claims.

1. A method for the detection of HIT in a mammalian patient, comprising:
(a) quantitatively measuring the presence of PF4-H complexes in a sample from a patient or the ability to form these complexes in vitro.
(b) detecting the presence of pathogenic autoantibodies specific for PF4-H complex in the patient sample; and
(c) detecting the presence of platelet activation in the patient,
the presence of PF4-H complexes, PF4-H autoantibodies, and platelet activation being indicative of the presence of HIT.

2. The method of claim 1, wherein the measuring step (a-c) is performed prior to treatment of the patient with heparin, during heparin treatment, and post-heparin treatment.

3. The method of claim 2, wherein the measuring step (a) is performed prior to the patient being treated with heparin, heparin being added to the patient sample before quantitatively measuring the presence of PF4-H complexes.

4. The method of claim 1, wherein the presence of PF4-H complexes is measured in a fluid sample from the patient.

5. The method of claim 1, wherein the step of measuring the presence of PF4-H complexes, of PF4-H autoantibodies, and of platelet aggregation occurs in one reaction solution.

6. The method of claim 1, wherein the steps of measuring the presence of PF4-H complexes, of PF4-H autoantibodies, and of platelet aggregation occur in at least one reaction solution.

7. The method of claim 6, wherein the steps of measuring the presence of PF4-H complexes, of PF4-H autoantibodies, and of platelet aggregation occur in three reaction solutions.

8. The method of claim 1, wherein steps (a), (b), and (c) occur simultaneously or sequentially.

9. The method of claim 3, wherein the presence of PF4-H complexes is detected using an antibody specific for the PF4-H complex, the antibody not recognizing heparin or PF4.

10. The method of claim 4, wherein the presence of PF4-H complexes is detected using more than one antibody specific for the PF4-H complex, the antibodies not recognizing heparin or PF4.

11. The method in claim 4, wherein a first antibody recognizes an epitope of PF4 not neutralized by the presence of heparin, and a second antibody recognizes an epitope of heparin not neutralized by the presence of PF4.

12. The method in claim 1, wherein the presence of heparin in the PF4-H complex is recognized by a heparin-binding compound which is not an antibody or fragment thereof.

13. The method of claim 1, wherein the pathogenic autoantibodies are detected by:
(a) creating a genetically engineered PF4 fusion molecule comprising a protein aggregation domain (PAD);
(b) adhering the fusion molecule to a solid support;
(c) contacting the adhered fusion molecule with the patient sample; and
(d) detecting and quantifying antibodies that bind to the adhered fusion protein.

14. The method of claim 13, wherein the bound autoantibodies are detected with an anti-human immunoglobulin.

15. The method of claim 13, wherein the bound autoantibodies are detected with anti-IgG.

16. The method in claim 13 wherein the bound antibodies are detected by Protein A or Protein G.

17. The method of claim 1, wherein the presence of platelet activation in the patient is determined by:
(a) immobilizing an antibody specific for PF4-H to a solid support,
(b) contacting the immobilized antibody with a sample from the patient, the platelets and/or microparticles in the sample binding to the antibody, thereby clustering activated Fc receptors on the bound platelet and/or microparticle membrane domains;
(c) treating the bound membrane with detergent to form a microparticle patch or a platelet patch; and
(d) detecting the presence of activated Fc receptor in the bound microparticle patch or in the bound platelet patch,
the activated Fc receptor being indicative of the presence of platelet activation in the patient.

18. The method of claim 17, wherein the activated Fc receptor is measured by detecting SyK protein kinase activity, an SyK protein kinase being activated when the Fc receptor is activated.

19. The method of claim 18, wherein the SyK protein kinase activity is measured by detecting enzymatic activity, fluorescence, or radioactive labels conjugated with SyK kinase or to its amino-terminal region containing the two SH2 binding domains.

20. The method of claim 1, wherein platelet activation is measured by:
(a) immobilizing CD41 antibodies specific for platelet CD41 to a solid support;
(b) contacting the immobilized CD41 antibodies with the patient sample;
(c) detecting the presence of microparticles from the patient sample adhered to the immobilized CD41 antibodies with:
(i) an anti-PF4-H antibody;
(ii) a phosphatidylserine (PS) binding molecule; and/or
(iii) an anti-tissue factor antibody,
the bound microparticles being indicative of platelet activation.

21. The method of claim 20, wherein the PS binding molecule comprises an anti-PS antibody or annexin V.

22. The method of claim 1, wherein platelet activation is measured by:
(a) immobilizing a peptide containing the SH2 domain sequence of SyK to a solid support;
(b) contacting the immobilized SH2 domain of SyK with the patient sample; and
(c) detecting bound platelet patches and bound microparticle patches by contacting the platelet and microparticles patches adhered to the SH2 domain of SyK with:
(i) an antibody recognizing PF4-H complexes;
(ii) an antibody recognizing PF4;
(iii) an antibody recognizing heparin; or
(iv) a non-antibody binding compound which recognizes heparin and/or PF4.

23. The method of claim 1, wherein the patient is a human.

24. The method of claim 1, wherein the patient sample is pre-treated with a low stringency detergent prior to steps (a-c).
